(19) United States
(12) Patent Application Publication
(10) Pub. No.: US 2026/0258457 A1
(43) Pub. Date: Sep. 3, 2026

(54) METHOD FOR PROCESSING A SEPARATION PRODUCT, CONTAINING CARBOXYLIC ACIDS, OF A SEPARATION PROCESS

(71) Applicants: K.D. PHARMA BEXBACH GMBH, Bexbach (DE); UNIVERSITÄT DES SAARLANDES, Saarbrücken (DE)

(72) Inventors: Christoph WITTMANN, Saarlouis (DE); Roxane RÖSNER, Düsseldorf (DE); Demian DIETRICH, Saarbrücken (DE); Michael KOHLSTEDT, Saarbrücken (DE); Sofija JOVANOVIC, Saarbrücken (DE); Felix HARION, Überherrn (DE); Gideon GIESSELMANN, Perl-Besch (DE); Denitsa SHOPOVA-GOSPODINOVA, Schönenberg-Kübelberg (DE); Stefan VIETEN, Kirkel (DE); Roberto FRONZONI, Homburg (DE)

(21) Appl. No.: 18/873,032

(22) PCT Filed: Jun. 9, 2023
(86) PCT No.: PCT/EP2023/065461

§ 371 Date: Dec. 9, 2024

(30) Foreign Application Priority Data
Jun. 9, 2022 (LU) LU502240

(51) Int. Cl. C12P 7/6427; C07C 51/42

(52) U.S. Cl. CPC C12P 7/6427; C07C 51/42

(57) ABSTRACT

A method for processing a separation product, containing carboxylic acids, of a separation process, in particular a process for separating fish oil and/or algae oil, in which method polyunsaturated hydrocarbons are produced at least from some of the carboxylic acids by microbes. Advantageously, the polyunsaturated hydrocarbons are or include omega-3 fatty acids and/or omega-6 fatty acids, preferably EPA, DHA and/or DPA. The microbes are or include yeast, fungus, bacteria and/or in particular marine protists, preferably microalgae. The yeast is preferably a yeast of the Yarrowia lipolytica species and the marine protist is preferably a microalga of the Schizochytrium genus, preferably Schizochytrium limacinum SR21. Biomass is produced from the microbes and includes polyunsaturated hydrocarbons.

The invention relates to a method of processing carboxylic acids.

US 2010/0166620 A1 discloses a system and a process for the production of fatty acid methyl esters, commonly referred to as “biodiesel”, obtained from a mixture of oils and grease of unknown composition which includes triglycerides, proteins or other matter in some organic form containing sufficient fatty acids to be converted into biodiesel.

It is the object of the invention to make carboxylic acids better utilizable.

The invention is achieved by a method of processing a separation product containing carboxylic acids from a, preferably industrial, separation method in which polyunsaturated hydrocarbons are produced by microbes at least from some of the carboxylic acids.

It has been found that, surprisingly, microbes can be used particularly efficiently for processing of the separation products. Among the materials that can be processed further by means of the method are by-products from industrial separation methods that have to date been typically disposed of. This advantageously not only reduces the costs of disposal and waste, but can also increase the yield of the separation method. This is especially true when it is an aim of the separation method to produce a product containing polyunsaturated hydrocarbons. The invention is found to be particularly advantageous when fish oil and/or algal oil are processed in the separation method.

The fish oil to be processed by the separation method, which is preferably an intermediate fish oil product that has been preprocessed, appropriately includes omega-3 fatty acid(s) at 30-80% by weight, preferably having at least one of the following contents: eicosapentaenoic acid (EPA) at 0-80% by weight, docosahexaenoic acid (DHA) at 0-70% by weight and/or docosapentaenoic acid (DPA) at 0-10% by weight.

The algal oil to be processed by the separation method, which is preferably an intermediate algal oil product that has been preprocessed, appropriately includes a content of omega-3 fatty acid(s) at 30-90% by weight, preferably having at least one of the following contents: eicosapentaenoic acid (EPA) at 0-85% by weight, docosahexaenoic acid (DHA) at 0-85% by weight and/or docosapentaenoic acid (DPA) at 0-10% by weight.

A starting fish oil from which the intermediate fish oil product is produced, in one embodiment of the invention, has a content of omega-3 fatty acid(s) of 10-50% by weight, preferably having at least one of the following contents: EPA at 0-25% by weight, DHA at 0-25% by weight and/or DPA at 0-7% by weight.

A starting algal oil from which the intermediate algal oil product is produced, in one embodiment of the invention, has a content of omega-3 fatty acid(s) of 10-80% by weight, preferably having at least one of the following contents: EPA at 0-70% by weight, DHA at 0-70% by weight and/or DPA at 0-5% by weight.

Appropriately, the starting fish oil and/or the starting algal oil has been produced by a distillation method, deacidification, ethylation/transesterification, hydrolysis, winterization, mechanical separation methods and/or supercritical fluid extraction, preferably produced from a raw oil.

In one embodiment of the invention, the intermediate fish oil product and/or the intermediate algal oil product are produced from the starting fish oil and/or the starting algal oil by distillation, deacidification, ethylation/transesterification, hydrolysis, mechanical separation methods, urea precipitation, chromatography, winterization, silver extraction methods and/or supercritical fluid extraction. The intermediate fish oil product and/or the intermediate algal oil product preferably has a content of ethyl esters of at least 50% by weight, preferably of at least 80% by weight.

The omega-3 fatty acid(s) mentioned are appropriately in the form of ethyl esters in the intermediate product, where the omega-3 fatty acid(s) are preferably in the form of ethyl esters to an extent of at least 50% by weight, preferably to an extent of at least 60% by weight, more preferably to an extent of at least 90% by weight.

In a particularly preferred configuration of the invention, the separation product is a by-product of a separation process for producing a target product containing omega-3 and/or omega-6 fatty acid(s), preferably EPA and/or DHA and/or DPA in high concentration.

In one embodiment of the invention, the target product that has especially been produced from fish oil and/or from algal oil has a content of omega-3 fatty acid(s) of >50% by weight, preferably of >80% by weight, more preferably of >90% by weight, preferably having at least one of the following contents: EPA at 0% to 99% by weight, DHA at 0% to 99% by weight and/or DPA at 0% to 20% by weight.

The target product appropriately has an EPA content of >80% by weight, preferably of >90% by weight, more preferably of >95% by weight, or a DHA content of >80% by weight, preferably of >90% by weight, more preferably of >95% by weight. In addition, the target product may have a DPA content of >50% by weight, preferably of >60% by weight, more preferably of >70% by weight.

The by-product appropriately has a content of saturated fatty acids of >10% by weight, preferably >15% by weight, more preferably >45% by weight. In one embodiment of the invention, the by-product has a content of polyunsaturated hydrocarbons, especially omega-3 and/or omega-6 fatty acid(s), preferably EPA, DHA and/or DPA, where the content is less than that of the target product, especially not more than 15% by weight, preferably not more than 10% by weight, more preferably not more than 5% by weight, of the target product. The content of polyunsaturated hydrocarbons, especially of omega-3 fatty acids, in the by-product is appropriately <15% by weight, preferably <10% by weight, more preferably <5% by weight.

In one configuration of the invention, the by-product, especially produced from fish oil and/or from algal oil, has at least one of the following contents: EPA at 0-25% by weight, preferably 0-15% by weight, DHA at 0-25% by weight, preferably 0-15% by weight, and/or DPA at 0-25, preferably 0-15% by weight.

The separation product is appropriately produced by:
a distillation method, especially using a distillation unit, preferably HPE (high-pressure extraction; for example as elucidated in WO2017005235A1) or short path distillation,
by SFE (supercritical fluid extraction) and/or
processed by a chromatography method, preferably SFC (supercritical fluid chromatography), high-performance liquid chromatography (HPLC), true moving bed chromatography (TMB) and/or simulated moving bed (SMB),
by a silver extraction method and/or
urea precipitation (urea complexation).

In one embodiment of the invention, the by-product produced by short path distillation has at least one of the following contents: EPA at 0-8% by weight, preferably 0-4% by weight, DHA at 0-5% by weight, preferably 0-3% by weight, and/or DPA, especially n-3-DPA, at 0-5% by weight, preferably 0-3% by weight.

The by-product produced by HPE has one of the following contents: EPA at 0-12% by weight, preferably 0-6% by weight, DHA at 0-7% by weight, preferably 0-4% by weight, and/or DPA, especially n-3-DPA, at 0-7% by weight, preferably 0-4% by weight.

The by-product produced by a silver extraction method has one of the following contents: EPA at 5-70% by weight, preferably 5-40% by weight, DHA at 5-70% by weight, preferably 5-40% by weight, and/or arachidonic acid at 0-12% by weight, preferably 0-6% by weight.

The by-product produced by urea precipitation has one of the following contents: EPA at 2-40% by weight, preferably 2-20% by weight, DHA at 2-40% by weight, preferably 2-20% by weight, DPA, especially n-3-DPA, at 2-20% by weight, preferably 2-10% by weight, and/or monounsaturated fatty acids at 5-60% by weight, preferably 5-30% by weight.

It has been found in a particularly advantageous manner that, by means of the microbes, it is possible to produce long-chain fatty acids, especially of chain length ≥12 carbon atoms, preferably chain length ≥20 carbon atoms, from short- or mid-chain fatty acids, especially of chain length <12 carbon atoms.

In one embodiment of the invention, the polyunsaturated hydrocarbons that are produced by the microbes are or comprise EPA, DHA and/or DPA.

In one configuration of the invention, the microbes are selected from the group of yeast, fungi, bacteria, protists, especially marine protists, preferably microalgae. Particular preference is given to using microbes selected from the group consisting of: Schizochytrium sp., Aurantiochytrium sp., Crypthecodinium cohnii, Isochrysis galbana, Phaeodactylum tricornutum, Dunaliella salina, Nannochloropsis oceanica, Chlorella vulgaris, Mucor circinelloides, Saccharomyces cerevisiae, Marine bacteria spp., Cyanobacteria spp., Myxobacteria spp.

In one embodiment of the invention, the microbes are or comprise a fat-dissolving yeast, preferably a yeast of the Yarrowia lipolytica species, more preferably a genetically modified yeast of the Yarrowia lipolytica species, for example Yarrowia lipolytica Af4.

In the studies by the inventors, it has been found that the yeast, especially of the Yarrowia lipolytica species, produces polyunsaturated hydrocarbons, especially DHA, from the carboxylic acids, especially the separation product.

For use in the method of the invention, it is also possible, for example, to employ the yeasts Candida tropicalis, Candida albicans, Debaryomyces hansenii and/or Trichosporon.

In a further embodiment of the invention, the microbes are or comprise a microalga of the Schizochytrium species, preferably Schizochytrium limacinum, more preferably Schizochytrium limacinum SR21.

It has also been found that the microalga of the Schizochytrium species produces polyunsaturated hydrocarbons, especially DHA, from the carboxylic acids, especially the separation product.

For use in the method of the invention, it is also possible to employ the following marine protists, especially microalgae or marine bacteria: Marinobacter, Oceanospiralles, Pseudomonas and/or Alkanivorax.

It has been found to be particularly advantageous to produce polyunsaturated hydrocarbons from the carboxylic acids, especially the separation product, by means of a combination of yeast and microalgae. In the case of production by means of yeast and microalgae, extracellular lipase activity of the yeast causes an emulsion effect that promotes the production of polyunsaturated hydrocarbons by the microalgae.

Compared to the yeast alone, the yield on utilization of the combination is about 4 times larger. Compared to the microalga alone, the yield is virtually doubled when the combination is used.

Production of unsaturated hydrocarbons by means of the microalga of the Schizochytrium limacinum species, especially Schizochytrium limacinum SR21, together with the yeast has been found to be particularly effective.

In one configuration of the invention, lipases are added for improved production of the polyunsaturated hydrocarbons by means of the microbes. The separation product is appropriately added to a reactor together with the microbes for production of the polyunsaturated hydrocarbons.

In one embodiment of the invention, biomass formed by the microbes is processed to a food or a pharmaceutical ingredient. The food is preferably fed to aquatic lifeforms, preferably fish, that are kept for production of fish oil, for example in a fish farm. In addition, the biomass may be fed to lifeforms in order to enrich their constituents or products (e.g. eggs) that are utilizable for nutrition with polyunsaturated hydrocarbons, especially omega-3 and/or omega-6 fatty acids. For example, it is possible to feed chickens with the biomass in order to achieve correspondingly elevated contents of polyunsaturated hydrocarbons in the meat and/or in the eggs produced.

The polyunsaturated hydrocarbons are appropriately extracted from biomass that has been produced by the microbes.

The invention further relates to biomass produced from microbes and including polyunsaturated hydrocarbons. The biomass appropriately includes yeast and/or microalgae, where the yeast is preferably of the Yarrowia lipolytica species and/or the marine protist is of the Schizochytrium species, preferably Schizochytrium limacinum SR21. The EPA, DHA and/or DPA content of the biomass is preferably at least 5 mg/g of dry weight.

The invention is elucidated in detail hereinafter by working examples and by tables and the appended graphs that relate to the working examples.

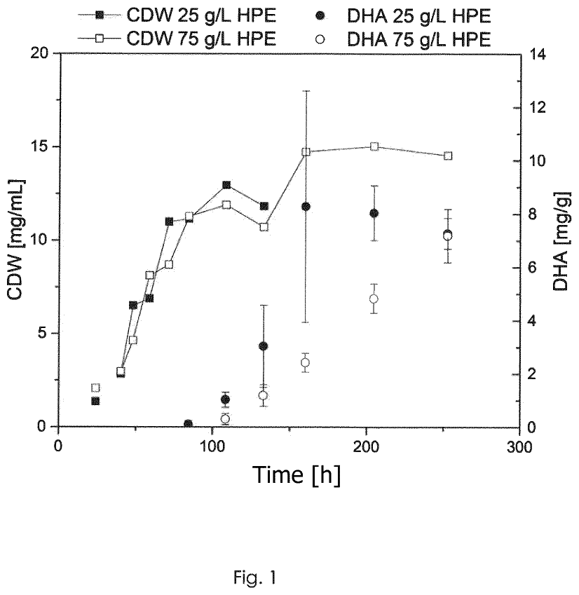

FIG. 1 shows, in a graph, a content of cell dry weight and a DHA content on performance of the method of the invention as a function of culture time.

### A. MATERIALS USED

A preprocessed fish oil that constitutes an intermediate product was processed by a separation method which is referred to hereinafter as “HPE” (high-pressure extraction) and has been described in publication WO2017005235A1. The intermediate product had the composition shown in table 1:

TABLE 1

| Fatty acid | Content [%] |
|---|---|
| C16:0 | 2.8 |
| C16:1 n7 | 1.1 |
| C16:3 n4 | 0.1 |
| C16:4 n1 | 0.3 |
| C18:0 | 3.8 |
| C18:1 n9 | 6.9 |
| C18:1 n7 | 3.3 |
| C18:2 n6 | 0.9 |
| C18:3 n6 | 0.2 |
| C18:3 n4 | 0.1 |
| C18:3 n3 | 0.6 |
| C18:4 n3 | 2.4 |
| C18:4 n1 | 0.4 |
| C20:0 | 0.8 |
| C20:1 n11 | 0.2 |
| C20:1 n9 | 2.7 |
| C20:1 n7 | 0.4 |
| C20:2 n6 | 0.1 |
| C19:5 | 0.4 |
| C20:3 n6 | 0.1 |
| C20:4 n6 | 2.3 |
| C20:4 n3 | 1.6 |
| C20:5 n3 | 39.5 |
| C22:0 | 0.3 |
| C22:1 n11 | 1.7 |
| C22:1 n9 | 0.6 |
| C21:5 n3 | 1.9 |
| C22:5 n6 | 0.4 |
| C22:5 n3 | 4.1 |
| C22:6 n3 | 14.7 |

After performance of the HPE, the target product had the following composition according to table 2:

TABLE 2

| Fatty acid | Content [%] |
|---|---|
| C16:0 | 0 |
| C16:1 n7 | 0 |
| C16:3 n4 | 0 |
| C16:4 n1 | 0 |
| C18:0 | 0.1 |
| C18:1 n9 | 0.1 |
| C18:1 n7 | 0 |
| C18:2 n6 | 0 |
| C18:3 n6 | 0 |
| C18:3 n4 | 0 |
| C18:3 n3 | 0.1 |
| C18:4 n3 | 0 |
| C18:4 n1 | 0 |
| C20:0 | 1.1 |
| C20:1 n11 | 0.3 |
| C20:1 n9 | 3.5 |
| C20:1 n7 | 0.5 |
| C20:2 n6 | 0 |
| C19:5 | 0 |
| C20:3 n6 | 0.5 |
| C20:4 n6 | 3 |
| C20:4 n3 | 2 |
| C20:5 n3 | 48.7 |
| C22:0 | 0.6 |
| C22:1 n11 | 2.6 |
| C22:1 n9 | 0.8 |
| C21:5 n3 | 2.5 |
| C22:5 n6 | 0.6 |
| C22:5 n3 | 5.9 |
| C22:6 n3 | 21.3 |

Polyunsaturated hydrocarbons have been produced as elucidated further down in the inventive manner by means of microbes from carboxylic acids from one of the fractions that do not constitute the target product of the separation method but are typically disposed of as waste.

The fatty acid composition of the fraction used, based on the totality of fatty acids present, is shown in table 3:

TABLE 3

| Fatty acid | Content in fraction [%] |
|---|---|
| C16:0 | 16.5 |
| C16:1 | 0 |
| C18:0 | 9.1 |
| C18:1 | 54.7 |
| C18:2 | 4.9 |
| C18:3 | 0.6 |
| C18:4 | 8.5 |
| C20:5 | 3.8 |
| C22:5 | 0 |
| C22:6 | 0 |
| Others | 1.8 |

### 1. Microorganisms

A strain of the oil-containing yeast Yarrowia lipolytica and the marine protist Schizochytrium limacinum, which is a microalga, were used for the culturing on the fraction according to table 3.
a) The following strain of the yeast Yarrowia lipolytica was used: Y. lipolytica, Po1h::SynPfaPptAf4 (Y. lipolytica Af4) [Gemperlein et al., 2019].
b) The following strain of the marine protist Schizochytrium limacinum was used: S. limacinum SR21 (ATCC MYA-1381) (renamed in 2007 to Aurantiochytrium limacinum).

### 2. Culturing

The following media were used for the culturing of Y. lipolytica and S. limacinum:

a) YNB Medium without Carbon Source

YNB medium was used in combination with a carbon source for the culturing of Y. lipolytica. All components were prepared as corresponding stock solutions in MilliQ water and either autoclaved at 120° C. for 20 min (ammonium sulfate) or sterile-filtered with a PES membrane filter of pore size of 0.2 μm (all others). The solutions were stored at room temperature (ammonium sulfate) or at 4° C. The concentrations are given in table 4.

TABLE 4

| Substance | | Concentration |
|---|---|---|
| calcium pantothenate | 400 | μg/l |
| inositol | 2000 | μg/l |
| niacin | 400 | μg/l |
| p-aminobenzoic acid | 200 | μg/l |
| pyridoxine hydrochloride | 400 | μg/l |
| riboflavin | 200 | μg/l |
| thiamine hydrochloride | 400 | μg/l |
| biotin | 2 | μg/l |
| folic acid (in 1M NaOH) | 2 | μg/l |
| boric acid | 500 | μg/l |
| copper sulfate hydrate | 25.54 | μg/l |
| potassium iodide | 100 | μg/l |
| manganese sulfate monohydrate | 303.08 | μg/l |
| sodium molybdate dihydrate | 200 | μg/l |
| zinc sulfate heptahydrate | 400 | μg/l |
| iron chloride hexahydrate (pH 1) | 200 | μg/l |
| potassium phosphate monobasic | 1 | g/l |
| magnesium sulfate heptahydrate | 500 | mg/l |
| sodium chloride | 100 | mg/l |
| calcium chloride dihydrate | 100 | mg/l |
| MES buffer at pH = 6.8 | 200 | mM |
| ammonium sulfate | 5 | g/l |

### b) Synthetic Seawater Medium

S. limacinum was cultured using a synthetic seawater medium with yeast extract as nitrogen source and a carbon source of the composition according to table 5. For mixed cultures of S. limacinum and Y. lipolytica, half the amount of salts was envisaged for the culturing (see values in brackets).

TABLE 5

| Substance | | Concentration |
|---|---|---|
| sodium chloride | 28.32 (14.16) | g/l |
| potassium chloride | 0.77 (0.385) | g/l |
| magnesium chloride hexahydrate | 5.48 (2.74) | g/l |
| magnesium sulfate heptahydrate | 7.39 (3.695) | g/l |
| calcium chloride | 1.1 (0.55) | g/l |
| sodium hydrogencarbonate | 0.2 (0.1) | g/l |
| yeast extract | 2-10 | g/l |

### c) Carbon Sources

The carbon source used for precultures was 10 g/l glycerol (stock concentration of 200 g/l; autoclaved at 120° C. for 20 min). Starting concentrations of 25 to 100 g/l of the fraction specified were used for main cultures. For each culture, the fraction was freshly sterile-filtered with a PES membrane filter having a pore size of 0.2 μm. The fraction was stored at 4° C. after the air in the bottle had been replaced by nitrogen in order to reduce the risk of oxidation.

### d) Culturing Conditions

The cells were cultured in 250 or 500 ml shaken flasks with 10% fill volume. The flasks were shaken in an orbital shaker (Multitron shaker, Infors HT, Bottmingen, Switzerland) at 200 or 230 rpm for S. limacinum/mixed cultures and Y. lipolytica at 28° C.

### 3. Methods

### a) Sampling

1.5 ml of culture medium is transferred from each triple flask to preweighed glass vials in duplicate. The exact volume is measured by gravimetry. The glass vials are centrifuged at 10 000×g and 4° C. for 3 minutes. A further 100 μl of culture medium is collected in a reaction vessel for pH measurement, microscopy and lipase assay.

### b) Supernatant

The supernatant, still containing residual oil, is removed from the cell pellet with a syringe fitted with a needle and filtered through a PES membrane filter (pore size 0.2 μm; HPLC quality) into a new reaction vessel. The oil-free supernatant is stored at −20° C. until further analysis by HPLC.

### c) Pellets

The remaining pellet in the glass vial is resuspended in 600 μl of hexane:water (1:6, v/v) and then centrifuged as elucidated above in order to remove adhering oil on the cell surface and on the glass wall of the vial. The water-hexane-oil mixture is discarded or, for non-quantitative residual fatty acid determination, the hexane-oil phase is collected in a separate glass vial and only the lower water phase is discarded. Both the hexane-oil phase and the wet cell pellets are stored at −20° C. until further processing.

### d) Determination of the Cell Dry Weight

The moist cell pellets are dried in a rotary vacuum concentrator (RVC 2-33 CDplus with infrared heating, Christ, Germany) at 30 mbar, 240 rpm and 40° C. for 30 minutes or until drying. Cell dry weight (CDW) is determined by gravimetry.

### e) Analysis of the Fatty Acids

The intracellular fatty acids are measured in each pellet sample (after CDW determination), as are the fatty acids in the residual HPE oil substrate (hexane-oil phase). The hexane-oil phase has to be dried for the next steps in a rotary vacuum concentrator (RVC 2-33 CDplus with infrared heating, Christ, Germany) at 30 mbar, 240 rpm and 40° C. for 30 min or until drying.

### f) Fatty Acid Methyl Ester (FAME) Preparation

The FAME preparation was conducted as follows. 15 μg of the methyl ester of heneicosapentaenoic acid (HPA) is added to each sample as internal standard for gas chromatography-mass spectroscopy (GCMS). 300 μl of a 50:50:2 methanol-toluene-sulfuric acid mixture (v/v/v) is added for the acid-catalyzed transesterification at 80° C. for 24 h. The reaction mixture is cooled on ice and the transesterification is stopped by adding 250 μl of a 0.5 M NH₄HCO₃ and 2 M KCl solution, followed by vortexing for 30 s. Phase separation is achieved by centrifugation at 10 000×g at room temperature for 3 min. 75 μl of the upper phase containing the FAME is transferred to a glass vial with an inlet for GCMS measurements.

### g) GCMS Measurement

Gas chromatography was conducted with an HP-6890 GC system (Hewlett-Packard, CA, USA) with a HP-88 column (30 m×250 μm×0.2 μm, Agilent Technologies) and helium (He 5.0) as carrier gas. The system was equipped with an injection system (series 7683B, Agilent Technologies, CA, USA) and an autosampler (series 7683, Agilent Technologies (CA, USA)). The injection volume was adjusted to 0.2 or 1.0 μl and the split mode was chosen as 10:1 or 5:1, according to the expected fatty acid concentration of the sample. Further parameters are given in table 6.

TABLE 6

| Inlet | | |
|---|---|---|
| Flow rate | 8.4 | ml/min |
| Pressure | 0.76 | bar |
| Temperature | 250° | C. |
| Analytes/sample | | |
| Flow rate | 1 | ml/min |
| Pressure | 0.76 | bar |
| Initial oven temperature | 110° C. for 1 minute | |
| Temperature gradient in oven | 4° | C./min |
| Final oven temperature | 240° | C. |

FAME were detected with a mass-selective detector (mass spectrometer, MS; 5973 Network Series from Agilent Technologies, CA, USA). The MS was set to a solvent delay of 5 minutes. The mass-selective detector was set to SCAN mode and measured the total ion current (TIC) from m/z 25 to m/z 500. For identification of the individual fatty acids, TIC and mass spectra were compared with the NIST08 mass spectra database (NIST/EPA/NIH Mass Spectral Library, 2008, version 2.0). In addition, retention times (RT) of individual fatty acid standard solutions (Sigma) were measured and compared for clarification with the RTs of fatty acids with unclear identity. MSD ChemStation G1701EA software was used for data analysis and system control.

### h) Quantification of Fatty Acids

The integration function of the MSD ChemStation G1701EA software was used to calculate the area beneath the curve for each peak. The results were paired with the retention times and exported to a table calculation file. The corresponding fatty acid was assigned to each RT measured. The amount of each FAME detected was then calculated in relation to the AUC signal of the HPA-ME standard. Fatty acid concentrations were calculated in relation to volume or CDW.

### 4. Results

The fraction with the fatty acid composition according to table 3 has been cultured in a reactor with the yeast Yarrowia lipolytica (cf. section 1. a)), with the marine protist Schizochytrium limacinum (cf. section 1. b)), and with a mixture of the yeast and the marine protist for 108 hours. Prior to culturing, the fraction contained no DHA, no DPA and 3.83% EPA.

As shown by table 7, culturing of the fraction with the yeast produced DHA. After culturing, the DHA content is 0.29% of the totality of the fatty acids. By culturing the fraction with the marine protist, such an amount of DHA has been produced that the content is 0.38%, and such an amount of DPA has been produced that the content is 0.07% by weight.

By means of culturing of the fraction with a mixed culture composed of the yeast and the marine protist, such an amount of DHA has been produced that the content is 6.34%, such an amount of DPA has been produced that the content is 1.96%, and such an amount of EPA has been produced that the content is 5.80%.

TABLE 7

| | | | | Y. lipolytica/ |
|---|---|---|---|---|
| | Content in | S. | Y. | S. limacinum |
| Fatty acid | fraction [%] | limacinum | lipolytica | mixed culture |
| C16:0 | 16.53 | 13.79 | 10.51 | 12.88 |
| C16:1 | 0 | 5.19 | 6.6 | 4.23 |
| C18:0 | 9.13 | 7.14 | 22.5 | 3.94 |
| C18:1 | 54.71 | 55.57 | 47.28 | 49.08 |
| C18:2 | 4.89 | 7.4 | 7.4 | 4.96 |
| C18:3 | 0.58 | 2.23 | 1.86 | 2.74 |
| C18:4 | 8.54 | 3.1 | 0.7 | 5.29 |
| C20:5 (EPA) | 3.83 | 1.44 | 0 | 5.8 |
| C22:5 (DPA) | 0 | 0.07 | 0 | 1.96 |
| C22:6 (DHA) | 0 | 0.38 | 0.29 | 6.34 |
| Others | 1.79 | 3.69 | 2.87 | 2.78 |

Table 8 shows the produced cell dry weight (CDW) of the biomass and the content of the totality of fatty acids in the cell dry weight and the content of DHA in the cell dry weight. It is found that the DHA content in the case of culturing with the marine protist is greater than that with the yeast. A significantly greater content is achieved in the case of culturing with the mixed culture.

TABLE 8

| | CDW | Totality of fatty | DHA |
|---|---|---|---|
| | [g/l] | acids [mg/g CDW] | [mg/g CDW] |
| Y. lipolytica | 7.7 | 507 | 8 |
| S. limacinum | 7.7 | 459 | 22 |
| Y. lipolytica & S. limacinum | 4.4 | 601 | 38 |
| (mixed culture) | | | |

The production of the greater EPA and DHA contents can be explained by a synergistic effect based on the fact that the yeast produces lipases that promote the production of EPA and DHA by the marine protist.

FIG. 1 shows, in a graph, the formation of biomass as a function of cell dry weight (CDW) as a function of culture time for a loading with 25 g/l and 75 g/l of the fraction (“HPE”) in the yeast culture, protist culture or mixed culture. In addition, the graph shows the change in the content of DHA for the two loadings as a function of culture time.

It is found that cell dry weight has risen continuously up to a culture time of 108 hours. For the loading of 25 g/l, the content of DHA has risen up to 10 mg/ml up to a culture time of 160 hours. For the loading of 75 g/l, the content of DHA has risen up to 10 mg/ml up to a culture time of 1250 hours.

In a further working example, oil that has been obtained from algae was processed by means of the separation method elucidated above. Polyunsaturated hydrocarbons have been produced in the inventive manner by means of microbes from carboxylic acids from one of the fractions that do not constitute the target product of the separation method. The results of a measurement of the yield of DHA in the processing of a fraction of algal oil and a fraction of fish oil are shown in table 9. It is found that the yeast is of better suitability for production of DHA from fish oil, while the DHA yield is greater in production by means of the microalgae for the fraction from algal oil.

TABLE 9

| DHA yield [mg/l] | HPE fraction of algal oil | | | HPE fraction of fish oil | | |
|---|---|---|---|---|---|---|
| Culture time [h] | 24 | 144 | 168 | 48 | 120 | 168 |
| S. limacinum | 115 | 232 | 245 | 55 | 57 | 48 |
| Y. lipolytica | 0 | 24 | 50 | 0 | 24 | 113 |

1-15. (canceled)

16. A method of processing a separation product containing carboxylic acids from a separation method, especially a method of separating fish oil and/or algal oil, comprising the step of producing polyunsaturated hydrocarbons at least from some of the carboxylic acids using microbes.

17. The method according to claim 16, wherein the polyunsaturated hydrocarbons are or comprise at least one of omega-3 fatty acids and omega-6 fatty acids.

18. The method according to claim 17, wherein the polyunsaturated hydrocarbons are EPA, DHA and/or DPA.

19. The method according to claim 16, wherein the microbes are or comprise at least one of yeast, fungi, bacteria and protists.

20. The method according to claim 19, wherein the microbes are marine protists.

21. The method according to claim 20, wherein the microbes are microalgae.

22. The method according to claim 19, wherein the yeast is a yeast of the Yarrowia lipolytica species.

23. The method according to claim 22, wherein the yeast is a genetically modified variant of the Yarrowia lipolytica species.

24. The method according to claim 20, wherein the marine protist is a microalga of the Schizochytrium species, preferably Schizochytrium limacinum SR21.

25. The method according to claim 24, wherein the marine protist is Schizochytrium limacinum SR21.

26. The method according to claim 16, wherein the product is a waste product of an industrial separation method.

27. The method according to claim 26, wherein the product is a waste product of a distillation method or a chromatography method.

28. The method according to claim 16, further including adding lipases for improving production by the microbes.

29. The method according to claim 26, further including adding the separation product to a reactor together with the microbes and optionally lipases for producing the polyunsaturated hydrocarbons.

30. The method according to claim 16, wherein the microbes form a biomass and the biomass is processed to give a food or a pharmaceutical ingredient.

31. The method according to claim 30, including feeding the food to aquatic lifeforms intended for production of fish oil.

32. The method according to claim 16, including extracting the polyunsaturated hydrocarbons from biomass formed by the microbes.

33. A biomass produced from microbes, the biomass comprising polyunsaturated hydrocarbons.

34. The biomass according to claim 33, wherein the biomass includes yeast, fungi, bacteria and/or protists, especially marine protists, preferably microalgae.

35. The biomass according to claim 34, wherein the biomass includes marine protists.

36. The biomass according to claim 35, wherein the biomass includes microalgae.

37. The biomass according to claim 33, wherein the biomass includes yeast of the Yarrowia lipolytica species and/or the marine protist of the Schizochytrium species, preferably Schizochytrium limacinum SR21.

38. The biomass according to claim 37, wherein the biomass includes Schizochytrium limacinum SR21.

39. The biomass according to claim 33, wherein the biomass has an EPA and/or DHA content of at least 5 mg/g of cell dry weight.
